(19) United States
(12) Patent Application Publication
(10) Pub. No.: US 2019/0314203 A1
(43) Pub. Date: Oct. 17, 2019

(54) PROTECTIVE EQUIPMENT

(71) Applicant: Alberto D'Orazio, Bradford (CA)

(72) Inventor: Alberto D'Orazio, Bradford (CA)

(21) Appl. No.: 16/385,105

(22) Filed: Apr. 16, 2019

Related U.S. Application Data
(60) Provisional application No. 62/658,058, filed on Apr. 16, 2018.

(51) Int. Cl. A61F 9/02; G02C 7/16; G02C 5/20

(52) U.S. Cl. CPC A61F 9/029; A61F 9/028; B01D 53/04; G02C 5/20; G02C 7/16

(57) ABSTRACT

A protective equipment apparatus for protecting a user is described. It includes an eye shield defining a nose ridge and a pair of lateral ends, the pair of lateral ends including a first end and a second end, and a first arm adjustably secured to the first end and a second arm adjustably secured to the second end. The apparatus further includes a first ear protector adjustably secured to a distal end of the first arm and a second ear protector adjustably secured to a distal end of the second arm and a nose respirator secured to the nose ridge of the eye shield where the nose respirator includes a carbon filter filtration element. Also disclosed is a protective device for protecting the face of a user and a replacement kit including a replacement filtration element for the nose respirator and replacement first and second ear protectors.

### FIELD OF THE INVENTION

The present specification relates to protective equipment, and more specifically to protective equipment to protect a user's face.

### BACKGROUND OF THE INVENTION

Protective equipment is desired or required in many situations. As an example, prudence often calls for users of heavy equipment and users in dusty environments to use protective equipment. As another example, government regulations often call for the use of protective equipment on construction job sites or in manufacturing areas.

Many items of protective equipment have been designed and produced to fulfill a great variety of functions. As an example, safety glasses have been designed to protect the eyes of a user. As another example, masks have been designed to filter air inhaled by a user. As another example, ear plugs have been designed to protect the ears of a user.

However, because of the great variety of functions for which protective equipment could be designed and the great variety of circumstances in which protective equipment could be used, improved protective equipment designs may be found desirable.

### SUMMARY OF THE INVENTION

In an embodiment of the present invention, there is provided an apparatus for protecting a user, comprising: an eye shield defining a nose ridge and a pair of lateral ends, the pair of lateral ends including a first end and a second end; a first arm adjustably secured to the first end and a second arm adjustably secured to the second end; a first ear protector adjustably secured to a distal end of the first arm and a second ear protector adjustably secured to a distal end of the second arm; and a nose respirator secured to the nose ridge of the eye shield.

In a further embodiment of the present invention, there is provided a protective device for protecting the face of a user, comprising in combination: safety glasses defining a nose arch, a first arm, and a second arm; a first ear plug adjustably secured to the first arm; a second ear plug adjustably secured to the second arm; and a nosepiece secured to the nose arch and configured to direct a nasal fluid flow through a filter.

In an embodiment of the present invention, there is provided a replacement kit for the aforementioned apparatus comprising a replacement filtration element, a replacement first ear protector and a replacement second ear protector.

In a further embodiment of the present invention, there is provided a replacement kit for the aforementioned protective device comprising a replacement filter, a replacement first ear plug and a replacement second ear plug.

### BRIEF DESCRIPTION OF THE DRAWINGS

The principles of the invention may better be understood with reference to the accompanying figures provided by way of illustration of an exemplary embodiment, or embodiments, incorporating principles and aspects of the present invention, and in which:

FIG. 1 is a perspective side view of protective equipment, according to an embodiment;

FIG. 2 is a perspective side view of the protective equipment of FIG. 1;

FIG. 3 is a perspective side view of the protective equipment of FIG. 1;

FIG. 4 is a perspective side view of the protective equipment of FIG. 1;

FIG. 5 is a front view of the protective equipment of FIG. 1;

FIG. 6 is a back view of the protective equipment of FIG. 1; and

FIG. 7 is a perspective view of an embodiment of a replacement kit.

### DETAILED DESCRIPTION OF THE EMBODIMENTS

The description that follows, and the embodiments described therein, are provided by way of illustration of an example, or examples, of particular embodiments of the principles of the present invention. These examples are provided for the purposes of explanation, and not of limitation, of those principles and of the invention. In the description, like parts are marked throughout the specification and the drawings with the same respective reference numerals. The drawings are not necessarily to scale and in some instances proportions may have been exaggerated in order more clearly to depict certain features of the invention.

This description relates to protective equipment to be provided to protect a user in a variety of circumstances or situations or events. This description relates to protective equipment provided to incorporate eye, nose, and ear protection in a single unit or modular unit.

This description relates to protective equipment which can be securely worn by a user during movement without incorporating bands, such as elastic bands, passing or secured around the user's head or neck. For example, embodiments are configured to be firmly secured to the head or face of a user during rapid movement of the head, including rapid movement of the head when the head is angled otherwise than vertically, without requiring the use of elastic bands passing around the user's head. In some circumstances, bands, such as elastic bands, passing over the head or neck of a user are uncomfortable or impractical or unsafe or difficult to put on.

This description relates to protective equipment incorporating eye and nose protection while limiting the impact of the nose protection on the functionality of the eye protection. For example, embodiments are configured to filter air received at the nose of a user while blocking vapor from the user's breath from fogging up the eye protection component or otherwise impacting the functionality of the eye protection. Embodiments incorporate a respirator to be held firmly against the face of a user to control the passage of air or vapor between a respiration zone defined by the equipment when in use and an ocular zone defined by the equipment when in use. In some embodiments, the protective equipment includes pads built into the nose piece, designed so that the nose piece will sit tightly against the cheek bone to keep vapor from escaping into the eye area of the protective equipment.

This description relates to protective equipment for filtering air inhalation without muffling or otherwise interfering with the ability of a user to speak.

This description relates to protective equipment incorporating adjustable ear protection. For example, embodiments include adjustable or flexible earpieces to allow a user to position earplugs comfortably when wearing the protective equipment.

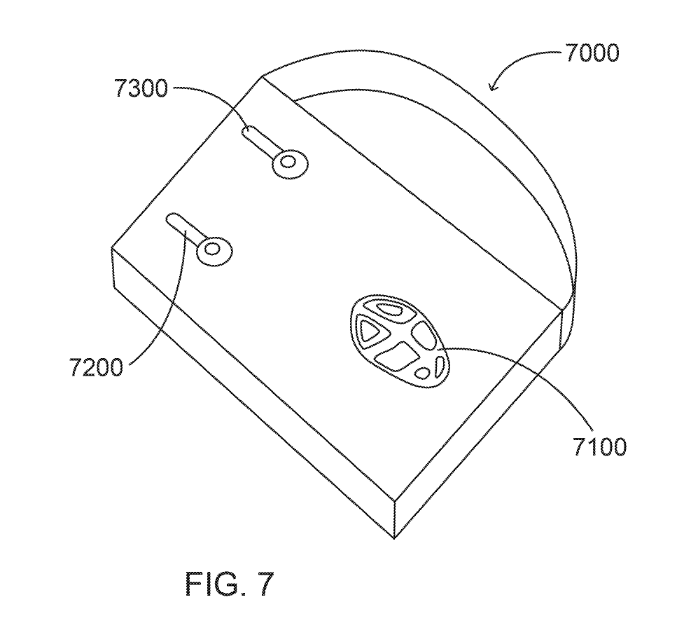

In some embodiments the protective equipment is a modular design. For example, in some embodiments one or more earpieces can be replaced with compatible earpieces. In some embodiments the nose protection may be interchangeable. For example, a nosepiece or nose respirator may be releasably secured to a nose ridge defined by an eye shield. In some embodiments, a filter component of the nose respirator may be replaceable or interchangeable. According to a further embodiment as shown in FIG. 7, a replacement kit 7000 may be provided which includes each of a replacement filter or filtration element 7100, a replacement first ear protector or earplug 7200, and a replacement second ear protector or earplug 7300.

Ear protection may include ear plugs formed of wax, foam, or other material, and may be designed to protect the ears from sound or environmental elements or both.

Eye protection may include an eye shield which is formed or partially formed of a transparent or semitransparent material such as glass, reinforced glass, plastic, or other material. An eye shield may be a singular piece or may be formed of multiple pieces. For example, the eye shield may be formed of a pair of eye lenses, may incorporate one or more frame components, and may include one or more extra components such as side shields.

Nose protection may include a nose cover provided to, when worn by a user, control the passage of fluid such as air between the user's nose and the ambient environment. Such nose protection may be formed of a plastic or other fluidly impervious material, formed to provide a fluid passage to be governed by a filtration element. The nose cover itself may integrally form the filtration element in some embodiments, while in other embodiments the filtration element may be a modular component of the nose protection.

In some embodiments, a filtration element is a carbon filtration element incorporating a volume of activated charcoal or biochar or other material to remove contaminants from a fluid passing through the filtration element. In some embodiments, a filtration element is a screen or other basic filter.

Manufacturing costs, desired lifespan, and reusability may be considerations in determining which elements of the protective equipment are formed integrally and which are formed modularly, as well as informing the choice of materials and construction technique and other design choices.

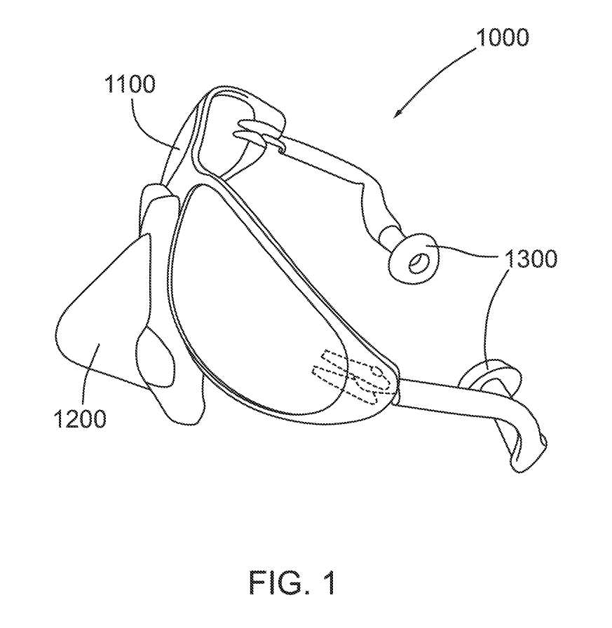

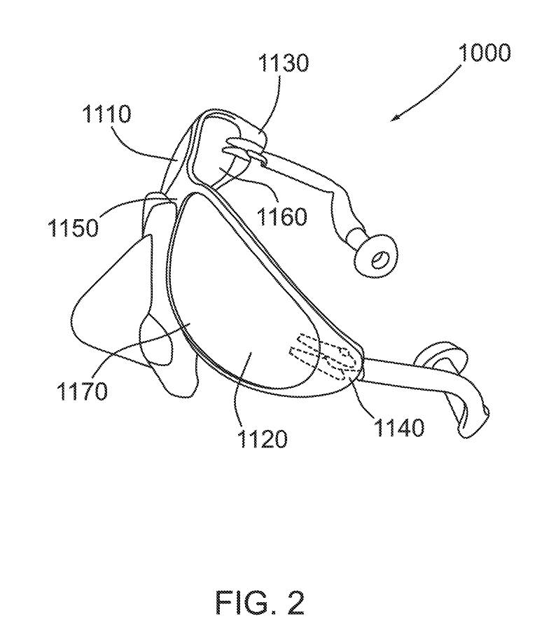

An embodiment is depicted in FIG. 1 and detailed further in FIGS. 2 to 4, in which a protection apparatus 1000 includes an eye shield 1100, a nose respirator 1200, and a set of ear protectors 1300.

As detailed particularly in FIG. 2, eye shield 1100 defines a first eye piece 1110 and a second eye piece 1120 for protecting the eyes of a user. In some embodiments, an eye shield may be a wrap-around shield to protect the outer corners of a user's eyes or may otherwise incorporate side shields. In some embodiments an eye shield may be a single piece of substantially transparent material to cover both of a user's eyes. In some embodiments, an eye shield may be two or more independent pieces of substantially transparent material, such as separate pieces for each eye or separate side shield pieces. In some embodiments, an eye shield may include one or more frame components.

Eye shield 1100 also defines a nose ridge 1150 and a pair of lateral ends 1140 and 1130, where the nose ridge 1150 and lateral ends 1140 and 1130 are understood to be sections or regions as indicated in FIG. 2 as opposed to points. As depicted, eye shield 1100 is a substantially flat piece of transparent material forming an inner surface 1160 and an outer surface 1170, and nose ridge 1150 and lateral ends 1140 and 1130 are partially defined by undulations or changes in the contour of eye shield 1100. In some embodiments the contour of an eye shield may be different from the contour of eye shield 1100 depicted. For example, in some embodiments an eye shield could be a rectangular shield designed to rest horizontally before a user's eyes, in which case the nose ridge may be the center of the bottom edge of the eye shield and not further defined by undulations in contour.

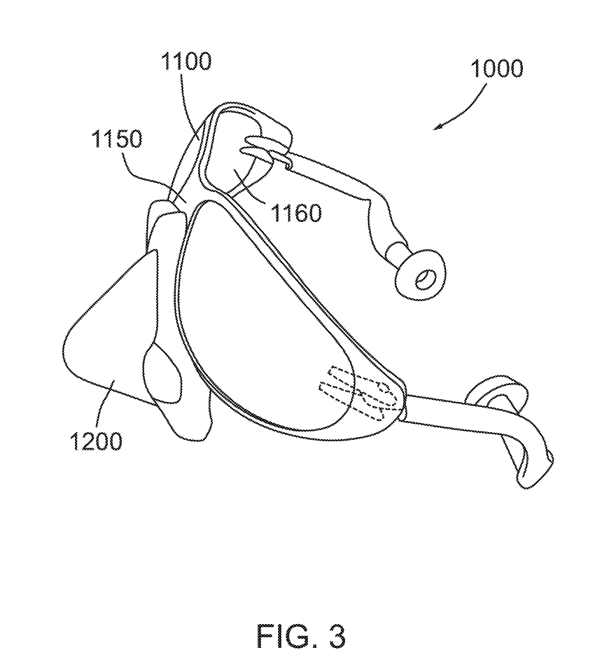

As detailed particularly in FIG. 3, nose respirator 1200 is secured to the nose ridge 1150 of eye shield 1100. Nose respirator 1200 is provided to filter air breathed by a user through the user's nose. Nose respirator 1200 is designed to sit snugly against the face of a user to impede the exchange of fluid between the nose and the ambient environment, to substantially direct the fluid through a passage defined in nose respirator 1200. By blocking the passage through the nose respirator 1200 with a filter, the nose respirator 1200 can be used to filter air breathed by a user through the user's nose. By covering the nose of a user and not the mouth, protection apparatus 1000 does not muffle or otherwise impede the user's voice, while also allowing the user to breath through a filter as desired or required. In some embodiments, a nose respirator may include or be formed of a nose base cover and a nose bridge cover, and in some embodiments the fluid passage may be formed in one or the other of the nose bridge and nose base covers. In some embodiments, the nose respirator may include a nose base cover and a nose bridge cover, and the nose base cover may be substantially formed of a filter providing a fluid passage, such as an interchangeable and replaceable modular filter designed to couple to receptive structure on the nose bridge cover to substantially form a nose respirator.

In order to minimize the effect of a user's breath on the transparency of eye shield 1100, such as to minimize condensation build-up, nose respirator 1200 is also designed to block the flow of fluid between the nose of a user and the inner surface 1160 of eye shield 1100. In addition to structural components of protection apparatus 1000 designed to be placed between a user's nostrils and eyes, nose respirator 1200 also controls the effect of breath on the eye shield by directing the external end of the defined fluid passage away from eye shield 1100, such as by having the fluid passage directed down through a nose base cover.

In some embodiments, the protective equipment includes one or more cheek pads or other sealing elements to assist in providing a fluid tight seal between the nose respirator and a user's face to assist in controlling the flow of fluid and directing fluid through the defined fluid passage. In some embodiments more than one fluid passage may be defined by nose respirator.

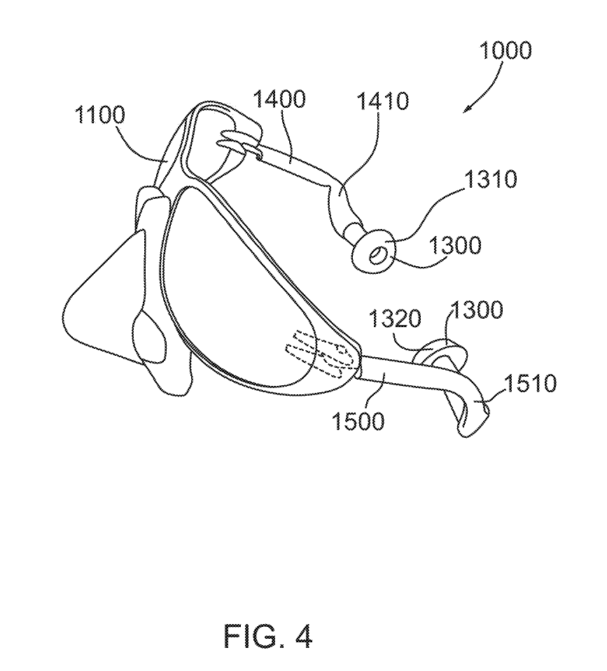

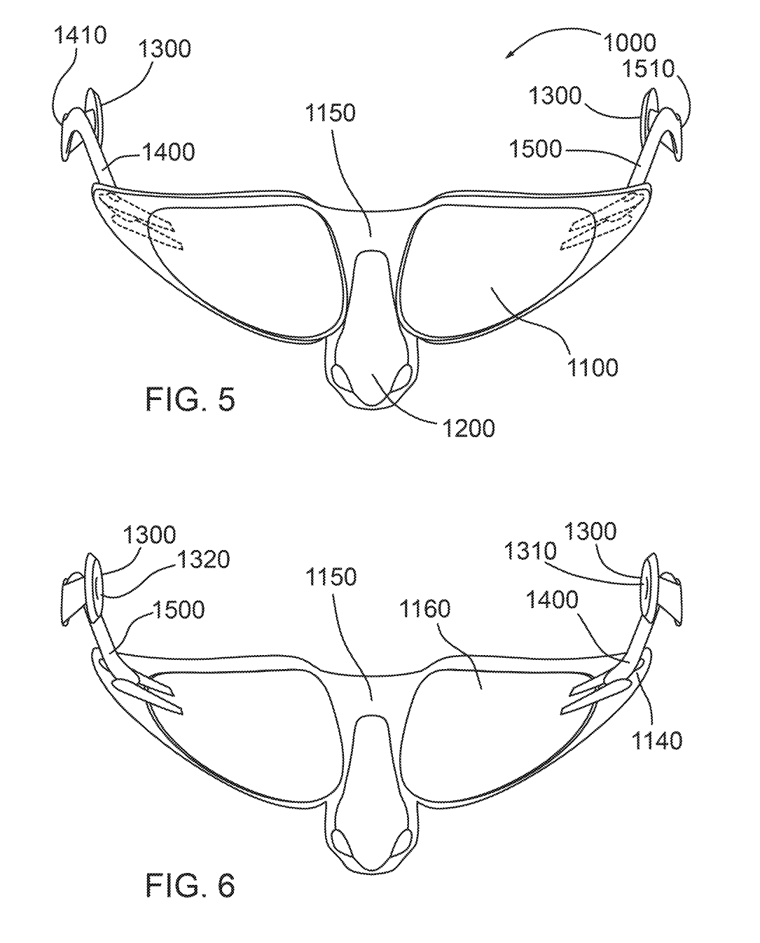

As detailed particularly in FIG. 4, protection apparatus 1000 also includes a first arm 1400 and a second arm 1500. Each arm is adjustably secured to eye shield 1100, the securing point allowing the arms at least one degree of freedom relative to the eye shield, such as to allow the arms to fold in in the usual manner. The arms may incorporate a number of features to allow ease of adjustment and use. For example, in some embodiments the arms may be extendable, such as telescoping. The arms are provided to interface with a user's ears to assist in firmly securing the protection apparatus 1000 to a user's head.

The set of ear protectors 1300 is adjustably secured to the arms. The set of ear protectors 1300 includes a first ear plug 1310 and a second ear plug 1320. First and second ear plugs 1310 and 1320 are each adjustably secured to the distal ends of first and second arms 1400 and 1500. The adjustable securement may be in a variety of ways, such as a pivotal securement to permit a user to adjust angles between the plugs and the arms. In some embodiments the ear protectors may be further adjustable, such as extendable to be compatible with deep-set ears. As depicted, ear protectors 1300 are ear plugs, however in other embodiments other protectors may be employed, such as muffs or covers.

As depicted, ear protectors 1300 substantially form the ends 1410 and 1510 of arms 1400 and 1500, where the ends 1410 and 1510 are understood to be the distal sections or regions of the arms 1400 and 1500 rather than the distal points of the arms. However, in other embodiments, the ear protectors may be secured further up the arms, such as to allow the arms to pass over the tops of a user's ears with the plugs extending down or back to be entered into the user's ear canals. As depicted in FIG. 1, ear protectors 1300 extend towards one another from the ends 1410 and 1510 of arms 1400 and 1500. However, in other embodiments, arms may be configured to pass over the tops of a user's ears, and ear protectors may need to include structure permitting the ear protectors to extend forward of the distal tips of the ends of the arms and towards the eye shield prior to extending inward to cover the ear canals.

In some embodiments, nose, ear, and eye protection can each provide more than one type of protection. As an example, in some embodiments eye protection may substantially function as safety glasses to protect the eyes from physical impact, and in some embodiments eye protection may also substantially function as goggles to isolate eyes from the environment. In another example, in some embodiments the nose respirator may be provided to filter air breathed through a user's nose, and in some embodiments the nose respirator may also be provided to shield or otherwise protect the nose from physical impact. In another example, in some embodiments ear protectors may be provided to protect a user's eardrums from sound vibrations, and in some embodiments ear protectors may also be provided to shield or otherwise protect ear drums or other ear components from physical impact.

A protection apparatus for providing eye, nose, and ear protection in a single unit or modular unit permits a design in which the components function together to hold the apparatus firmly to a user's head when in use and in which the components do not degrade one another's functionality. In addition, the provision of a single unit or modular unit could be convenient for some users.

Various embodiments of the invention have been described in detail. Since changes in and or additions to the above-described best mode may be made without departing from the nature, spirit or scope of the invention, the invention is not to be limited to those details but only by the appended claims.

What is claimed is:

1. An apparatus for protecting a user, comprising:
an eye shield defining a nose ridge and a pair of lateral ends, the pair of lateral ends including a first end and a second end;
a first arm adjustably secured to the first end and a second arm adjustably secured to the second end;
a first ear protector adjustably secured to a distal end of the first arm and a second ear protector adjustably secured to a distal end of the second arm; and
a nose respirator secured to the nose ridge of the eye shield.

2. The apparatus of claim 1, wherein the nose respirator includes a carbon filter filtration element.

3. The apparatus of claim 2, wherein the nose respirator includes a nose bridge cover and a nose base cover, and the carbon filter filtration element is included in the nose base cover.

4. The apparatus of claim 1, wherein the eye shield further defines an inner surface and an outer surface, and the nose respirator is configured to inhibit fluid communication between the nose and the inner surface of the eye shield.

5. The apparatus of claim 1, wherein the first arm and the second arm are telescoping arms.

6. The apparatus of claim 1, wherein the first and second ear protectors are pivotally secured to the first and second arms.

7. The apparatus of claim 1, wherein the first and second ear protectors are ear plugs.

8. The apparatus of claim 1, wherein the first and second ear protectors are adjustably secured to the distal tips of the distal ends of the first and second arms.

9. The apparatus of claim 1, wherein at least one of the filtration element, the first ear protector, or the second ear protector are replaceable.

10. A protective device for protecting the face of a user, comprising in combination:
safety glasses defining a nose arch, a first arm, and a second arm;
a first ear plug adjustably secured to the first arm;
a second ear plug adjustably secured to the second arm; and
a nosepiece secured to the nose arch and configured to direct a nasal fluid flow through a filter.

11. The protective device of claim 10, wherein the safety glasses further define an inner surface and an outer surface, and the nosepiece is further configured to inhibit the nasal fluid flow from passing over the inner surface of the safety glasses.

12. The protective device of claim 11, wherein the nosepiece is further configured to inhibit the nasal fluid flow from passing over the outer surface of the safety glasses.

13. The protective device of claim 10, wherein the filter is configured to remove a first category of particulate matter from the nasal fluid flow.

14. The protective device of claim 10, wherein the first and second ear plugs are adjustably secured to the first and second arms via first and second pivot connections.

15. The protective device of claim 10, wherein at least one of the filter, the first ear plug or the second ear plug are replaceable.

16. A replacement kit for the apparatus of claim 1 comprising a replacement filtration element, a replacement first ear protector and a replacement second ear protector.
